(19) United States
(12) Patent Application Publication
(10) Pub. No.: US 2020/0179642 A1
(43) Pub. Date: Jun. 11, 2020

(54) DEVICE, METHOD AND SYSTEM FOR VERTIGO THERAPY

(71) Applicant: The Board of Regents of the University of Texas System, Autin, TX (US)

(72) Inventors: Maria Stephanie JARDELEZA, San Antonio, TX (US); Henrick Youval KRIGEL, San Antonio, TX (US)

(73) Assignee: The Board of Regents of the University of Texas System, Austin, TX (US)

(21) Appl. No.: 16/316,805

(22) PCT Filed: Jul. 6, 2017
(86) PCT No.: PCT/US2017/040811

§ 371 Date: Jan. 10, 2019

Related U.S. Application Data
(60) Provisional application No. 62/360,547, filed on Jul. 11, 2016.

(51) Int. Cl. A61M 21/02; A61B 5/11; A61B 5/00

(52) U.S. Cl. CPC A61M 21/02; A61B 5/1116; A61B 5/4023; A61M 2021/0027; A61M 2209/088; A61M 2210/06; A61M 2205/502; A61B 5/486

(57) ABSTRACT

Disclosed herein are devices, methods, and systems for monitoring the position of a subjects head and/or provide instruction to bring the subject's head into a desired position, to perform a desired movement, or a series of head positions and/or movements.

This Application claims priority to U.S. Provisional Patent Application 62/360,547 filed Jul. 11, 2016, which is incorporated herein by reference in its entirety.

The present invention relates to medical devices, methods, and systems. In particular embodiments the devices, methods, and systems monitor the position of a subjects head and provide instruction to bring the subject's head into a desired position, to perform a desired movement, or a series of head positions or movements for treating vertigo.

### BACKGROUND

Dizziness is a term that describes sensations such as faintness, wooziness, weakness, or unsteadiness. Dizziness affects at least 90 million Americans (27% of the population) at least once in their lifetime, and is a common reason for falls and physician visits, especially in the elderly. Dizziness that creates the false sense that a person or their surroundings are moving or spinning is called vertigo (Mayo Clinic, Diseases and Conditions, Dizziness, Aug. 11, 2015). The most common cause of vertigo is Benign Paroxysmal Positional Vertigo (BPPV), which causes an intense and brief false sense of spinning or moving. BPPV can be triggered by a rapid change in head movement that can be as benign as turning over in bed or sitting up. BPPV diagnoses is widely achieved through observation of a patient performing the Dix-Hallpike maneuver, but instruments providing guidance for the movement of the patients head in the Dix-Hallpike maneuver have not been described.

Vestibular rehabilitation therapy is the only non-pharmacologic management available for vertigo. It involves coordinating a multidisciplinary team composed of a physician, an occupational therapist, and a physical/vestibular therapist. Head positioning exercises are often part of the treatment for vertigo. These head positioning exercises are created in the office with the medical team and given to the patient to “perform” at home. The exercises have the patient move their head through a specific series of maneuvers. However, the head positioning treatments can easily be ineffective when performed without real-time observation and guidance from a medical care provider, as it is very difficult for a patient to determine if they are positioning their head in a manner that is effective for the treatment. Thus, the head positioning treatments must also often be done under the supervision of a physician, which can be expensive, time consuming, and difficult to obtain to immediately treat a vertigo spell.

Currently, there is no head positioning device that helps a subject and/or medical care provider optimize/maintain/monitor the subject's head positioning accurately. Thus, there is a need for devices and methods for monitoring and instructing the subject regarding the effective position of the head for complex or difficult treatment regimes.

### SUMMARY

Disclosed herein is a device, system, and method to monitor the head position of a subject and provide instruction to the subject in order to diagnose or treat the subject. In certain aspects the subject will be suspected of or diagnosed with vertigo. The device can be configured to provide instructions and monitoring for positioning a subject's head in a desired position, performing a desired movement, or performing a series movements and achieving various head positions.

In some instances, the head positioning device, system, and/or method can be used to provide and monitor vestibular rehabilitation therapy. The system allows the therapy to be performed by the subject at a location outside of a medical care facility or without real-time supervision by a medical care provider. In some instances, it can serve to provide feedback to the subject, a medical care provider, or a medical team regarding the therapeutic regime performed or the results obtained by positioning exercises.

Certain embodiments are directed to a device comprising at least one sensor, a microprocessor, a user interface, an electronic storage medium (e.g., EEprom data memory), and a controller. In certain aspects a sensor can be a position sensor (e.g., accelerometer), a camera, or a position sensor and a camera. The positional sensor can be configured to provide positional information related to a subjects head. A position sensor is configured to detect the position of the head in three dimensions, including the tilt of the head forward and backward, the tilt of the head side to side, and the rotation of the head. A head position can be based on the degree of tilt or rotation from a normal position, e.g., sitting straight and facing forward.

In certain aspects, the device, or a portion of the device, is configured to be wearable or to attach to a wearable component, housing, or cradle. In a further aspect the device is configured to be worn on or attached to the head. A camera can be provided or worn and configured to provide images of the eye and eye movements. If a camera is included it is configured to detect movement of the eye. Eye movement can be detected and used to monitor, diagnose, or detect vertigo.

In some instances, the device comprises a user interface that can include a microphone, a speaker, a touchscreen, a push button, a switch, or various combinations thereof for entering data and controlling the system. In a particular aspect the user interface can include a touchscreen, a voice activated controller, or include both a touchscreen and voice activated controller.

The device can be programmed to monitor the position of a portion of a subject's body to which the device is connected, in particular the subject's head. In some aspects, the device is programmed to monitor and implement a treatment involving positioning or moving part of a subject's body. In certain aspects the movement and positioning of the head is used to manipulate the position of a subject's ear canal. In certain aspects, the treatment is a treatment for vertigo.

The user interface can provide for the receipt or transmission of information, such as instructions for a task or confirmation that a task or treatment has been performed. In certain aspects, the device will display the task or treatment to be performed, and will provide real-time instruction regarding how to complete or progress in completing the task or treatment. In some aspects, the real-time instruction can be provided visually, audibly, or both visually and audibly. The sounds can include, but are not limited to, voice commands, videos, illustrations, beeps, or other audible signals.

In certain respects the device can be programmed with various routines and programs. In certain aspect 1, 2, 3, 4, 5 or more treatments or routines can be programmed. In some aspects, each treatment is programmable on a per patient basis. A health care provider can select at least one treatment of routine for a patient.

In certain aspects, the device is configured to record user input or compliance over a period of time. In some aspects, the data can be stored locally on the device for a period time until downloaded or transmitted to a receiving device or server. In some instances, the device can be configured to transmit data when a communication connection is available. In some instances, the device can be connected to or connectable to a network. In some aspects, the device can be configured to store data locally until a communication connection is available, e.g., SD memory card. In certain aspects, the data can be stored on a removable storage that is periodically removed, data downloaded, and re-inserted.

Certain embodiments are directed to devices for monitoring a subject. In certain aspects, a head position device is used to monitor the positioning of a subject's head. In certain aspects, a head position device comprises a three-axis accelerometer sensor (position sensor) mounted inside a device that is worn by a subject, in particular the device can be worn on the head. In certain aspects the device to be worn by the subject is in the form of a band, a clip, a hat, eye wear, headphones, ear bud(s), or the like. The device can also be attached to the skin of the subject, e.g., by using an adhesive. In certain aspects the device can be affixed to the subjects skin with a removable adhesive.

In certain aspects, the head position device is in communication with a base unit that comprises a microprocessor based display, a data logging unit, and a data storage device. In certain aspects, the display and data logging device is configured as a touchscreen. In certain embodiments the data storage device is EEprom data memory. In some aspects, after setting the head in the desired position, the head position is registered electronically, and the values of the three axis are saved in the unit's memory for reference.

In some instances, the storage medium is capable of storing more than one spatial orientation of the sensor (target position) under a unique identifier. In other aspects the processor is capable of determining the difference between the real-time spatial orientation of the sensor and a target position—indicating to the user if the positions are similar or not. A user interface can also be capable of receiving input from a user or a sensor (i) when the spatial orientation of the sensor is to be stored under a unique identifier (e.g., programming mode), and (ii) when the difference is to be determined by the processor between (a) the real-time spatial orientation of the sensor and (b) the target position (e.g., therapy mode). A speaker or sound system capable of producing sound or speech to indicate if there is or is not a difference in the real-time spatial orientation of the sensor and the target position. The sensor(s) is capable of electronically or wirelessly coupling to one or more of the storage, the processor, and/or the user interface display directly or indirectly.

In some instances, the display is capable of indicating if there is or is not a difference in the real-time spatial orientation of the sensor and the stored orientation of the sensor under a unique identifier (target position), i.e., if the treatment position has been attained. In some instances, the sound system and/or display is capable of indicating the direction in which the body part needs to be moved to attain a certain position. In certain aspects an audible indicator with verbal instructions or an audible frequency changes as you move closer to or further away from a programmed position. For example the frequency can shorten as you move closer to a target position and lengthen as you move away from a target position. In some instances, more than one spatial orientation of the sensor are stored under unique identifiers and are capable of being stored as sequential orientations to be achieved in one or more regimen stored in the memory under a unique identifier(s). In some instances, the sound system and/or display are capable of indicating that the sensor real-time position is within a certain difference between the real-time position of the sensor and the stored orientation of the sensor within a stored regimen. The sound system and/or display are capable of then, or at the same time, indicating the direction the body part needs to move to attain the next sequential orientation of the sensor stored under a unique identifier in the regimen stored under a unique identifier. In some instances, a timer is capable of timing the amount of time the sensor's real-time position is within a target position. In some instances, the sound system and/or display is capable of indicating that the sensor real-time position is within a certain target position for at least an amount of time. The sound system or display is then capable of indicating the direction the body part needs to move to be within the next target position of a regimen or that the regimen has been completed. In some instances, the device is capable of storing data regarding if and/or when the sensor real-time position is within range of a target position. In some instances, the device is capable of transmitting data regarding if or when the sensor real-time position was within a certain range of a target position. In some instances, the device is capable of displaying a representation of the orientation of the body part needed to place the sensor within range of the target position. In some instances, the sound system is capable of producing voice commands to indicate if there is or is not a difference in the real-time spatial orientation of the sensor and the stored orientation of the sensor under a unique identifier.

In some instances, the target position corresponds with a position of the body part that is to be achieved during a medical treatment or diagnostic. In some instances, the medical treatment or diagnostic is to be performed outside of the real-time supervision of a medical care provider. In some instances, the part of the subject's body is the subject's head. In some instances, the medical treatment or diagnostic is to treat or diagnose vertigo.

Certain aspects are directed to a system for tracking the orientation of a body part. In some instances, the system includes one or more sensors capable of determining the one, two, or three dimensional spatial orientation of the sensor. The sensor can be capable of being removably attached to a part of the subject's body. The system can include a storage medium capable of storing more than one spatial orientation of the sensor under unique identifiers and a processor capable of determining the difference between the real-time spatial orientation of the sensor and a stored orientation (target position) of the sensor stored under a unique identifier. The system can also include a user interface capable of receiving input from and/or transmitting output to a user. The user or a programmer can indicate when the spatial orientation of the sensor is to be stored in storage under a unique identifier for the stored orientation. The user interface can signal or indicate when a target position has been reached. The user interface can include a sound system capable of producing sounds to indicate if there is or is not a difference in the real-time spatial orientation of the sensor and the target position. There can be one or more displays. In some instances, the display is capable of displaying an indication if there is or is not a difference in the real-time spatial orientation of the sensor and the stored orientation of the sensor under a unique identifier. In some instances, the system is capable of transmitting data regarding if or when the sensor real-time position is within a target position. In some instances, the system is capable of displaying a representation of the orientation of the body part needed to place the sensor within certain difference or less between the real-time position of the sensor and the target position. In some instances, the sound system is capable of producing voice commands to indicate if there is or is not a difference in the real-time spatial orientation of the sensor and the stored orientation of the sensor under a unique identifier.

In some instances, the stored orientation of the sensor under a unique identifier corresponds with a position of the body part that is to be achieved during a medical treatment or diagnostic. In some instances, the medical treatment or diagnostic is to be performed outside of the real-time supervision of a medical care provider. In some instances, the part of the subject's body is the subject's head. In some instances, the medical treatment or diagnostic is to treat or diagnose vertigo in the subject. In some instances, the sensor is electronically or wirelessly coupled to the storage medium, the processor, the user interface, the sound system, and the display directly or indirectly.

Also disclosed herein are methods of using the device and system disclosed herein. In some aspects, a method of programming any one of the devices or systems is disclosed. In some instances, the method includes in any order (i) orienting the sensor into a first desired spatial orientation, (ii) electronically or wirelessly coupling the sensor to the processor, storage medium, and user interface directly or indirectly, (iii) providing input through the user interface regarding when the first spatial orientation of the sensor is to be stored, (iv) providing or indicating through the user interface the unique identifier the first spatial orientation of the sensor is to be stored under, wherein the first spatial orientation of the sensor under the unique identifier is stored in the storage. In some instances, the method further includes storing more than one spatial orientation of the sensor by repeating the steps for every spatial orientation to be stored: orienting the sensor into the desired spatial orientation; providing input from a user through the one or more user interface regarding when the spatial orientation of the sensor is to be stored in the storage medium; providing or indicating through the user interface the unique identifier the spatial orientation of the sensor is to be stored under; wherein the spatial orientation of the sensor under the unique identifier is stored in the storage medium. In some instances, the method includes programming a series of spatial orientations of the sensor as sequential orientations to be achieved during a regimen stored in the memory under unique identifier. In certain aspects the regimen includes information regarding how long to hold the position.

Certain aspects are directed to a method of monitoring the position of a subject's body part relative to at least one pre-programmed desired orientation of the subject's body part by using the any one of the devices or systems disclosed herein. In some instances, the method includes in any order: removably attaching a sensor on the subject's body part to be monitored; electronically or wirelessly coupling the sensor directly or indirectly to the processor, storage medium, and user interface, wherein the storage medium has been preprogrammed to contain at least one orientation of the sensor associated with a desired orientation of the subject's body part; optionally using the user interface to select the pre-programmed desired orientation of the sensor associated with a desired orientation of the subject's body part to be used by the processor in determining a difference between the real-time spatial orientation of the sensor and the pre-programmed desired orientation of the sensor; electronically or wirelessly coupling the processor directly or indirectly to at least one display or at least one sound system so that it indicates if there is or is not a difference in the real-time spatial orientation of the sensor and the pre-programed desired orientation of the sensor, wherein monitoring indication from the at least one display or at least one sound system provides information regarding the position of a subject's body part relative to the at least one pre-programed desired orientation of the subject's body part. In some instances, the position of the subject's body part is monitored sequentially relative to more than one pre-programed desired orientations of the subject's body part. In some instances, the more than one pre-programmed desired orientations of the subject's body part correspond with orientations of the body part in a medical treatment or diagnostic regime. In some instances, the part of the subject's body is the subject's head. In some instances, the treatment or diagnostic regime is to treat or diagnose vertigo in the subject.

Other embodiments of the invention are discussed throughout this application. Any embodiment discussed with respect to one aspect of the invention applies to other aspects, of the invention as well and vice versa. Each embodiment described herein is understood to be embodiments of the invention that are applicable to all aspects, of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used in this specification and claim(s), an accelerometer is an electromechanical sensor that can detect and/or measure physical acceleration experienced by an object. In some aspects, an accelerometer can measure any movement or vibration of an object.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of one example of a head position monitor 110, 112 and a display/user interface 100 electronically or wirelessly coupled to the head position monitor.

FIG. 2. Schematic of one example of a display and a user interface device coupled to a head position monitor.

FIG. 3. Flow chart for a preview scheme for the Epley maneuver.

FIG. 4. Flow chart for a performance scheme for the Epley maneuver.

FIG. 5. Flow chart for a performance scheme for the Dix-Hallpike maneuver.

### DESCRIPTION

Embodiments of the invention can be applied in a variety of settings to monitor a subject's head position, monitor a treatment regimen involving positioning of a subject's head or movement of a subject's head, or providing instruction to a subject or a medical care provider regarding positioning of a subjects head to be compliant with a desired head position. Certain embodiments describe a treatment for vertigo.

A subject's inability to determine if their head position or head movement is in compliance with a treatment position or regimen has been shown to be a prominent concern for home treatment of diseases and conditions such as, but not limited to, vertigo. Several factors contribute to patient non-compliance, which included dizziness or vertigo symptoms in a patient, the lack of a trained medical care provider present to provide instruction to assist the subject in positioning or moving their head to be in compliance with the treatment, and difficulty in remembering the exact positioning or movement of the head required by the treatment.

Embodiments are directed to a device, method, and system for monitoring compliance with treatment regimens involving head position or head movement. A treatment regimen can be prescribed by a physician or other qualified medical personnel. In certain embodiments a subject is provided a description of the treatment and specific instructions concerning each treatment. In certain aspects, unique identifiers for the treatment can be provided. In certain aspects, a device is programmed to use a unique identifier to identify a particular treatment and/or a particular subject.

In certain embodiments the treatments include an Epley maneuver, a Lempert maneuver, and/or a deep hanging maneuver. In other embodiments diagnostic procedures can include Dix-Hallpike maneuver.

The Epley maneuver or repositioning maneuver is a maneuver used to treat benign paroxysmal positional vertigo (BPPV) of the posterior or anterior canals. The maneuver allowing free floating particles from the affected semicircular canal to be relocated, using gravity, back into the utricle, where they can no longer stimulate the cupula, therefore relieving the patient of bothersome vertigo. A current version of the maneuver called the “modified” Epley does not include vibrations of the mastoid process originally indicated by original maneuver. The following sequence of positions describes the Epley maneuver: (i) The patient begins in an upright sitting posture, with the legs fully extended and the head rotated 45 degrees towards the side in the same direction that gives a positive Dix-Hallpike test. (ii) The patient is then quickly and passively forced down backwards by the clinician performing the treatment into a supine position with the head held approximately in a 30-degree neck extension (Dix-Hallpike position), and still rotated to the same. (iii) The clinician observes the patient's eyes for “primary stage” nystagmus. (iv) The patient remains in this position for approximately 1-2 minutes. (v) The patient's head is then rotated 90 degrees to the opposite direction so that the opposite ear faces the floor, all while maintaining the 30-degree neck extension. (vi) The patient remains in this position for approximately 1-2 minutes. (vii) Keeping the head and neck in a fixed position relative to the body, the individual rolls onto their shoulder, rotating the head another 90 degrees in the direction that they are facing. The patient is now looking downwards at a 45-degree angle. The eyes should be immediately observed for “secondary stage” nystagmus and this secondary stage nystagmus should be in the same direction as the primary stage nystagmus. The patient remains in this position for approximately 1-2 minutes. Finally, the patient is slowly brought up to an upright sitting posture, while maintaining the 45-degree rotation of the head. The patient holds sitting position for up to 30 seconds. The entire procedure may be repeated two more times, for a total of three times. During every step of this procedure the patient may experience some dizziness.

The Lempert maneuver is a maneuver used for horizontal vestibular canal treatment. The Lempert maneuver includes: (i) The person lies on his back 30 sec. (ii) Turns on the healthy side hand under the head supporting it horizontally. Position 30 sec or until rotation stops. If problems are in this, the tester helps keeping head immobile. (iii) Turns on the belly, the forehead lowest for 30 seconds. (iv) Turns on the problem side head supported horizontally hands under the head for 30 seconds. (v) Subject sits up and waits until equilibrium is steady and the sight clear. (vi) Subject sits in a steady chair hands on armrests for 2 min.

The deep hanging maneuver (DHM) consists of 4 steps with intervals of at least 30 seconds. (i) The subject is assisted from a long-sitting position into a supine position. (ii) The head is extended backwards by 30 degrees. (iii) The supine position is maintained while their head is flexed forward 45 degrees above the horizontal plane. (iv) the patient is returned to the sitting position. In position 1, the otoconia lie near the ASC ampulla. In position 2 (head-hanging position), both ASC's are inverted with their ampullas superior and their non-ampullary endings medial and inferior. Otoconia migrate due to their weight towards the apex of the ASC. In position 3 (chin to chest), gravity facilitates further migration towards the common crus. Finally, in position 4, the patient sits up with head tucked in. This last step allows otoconia to move through the common crus and into the utricle.

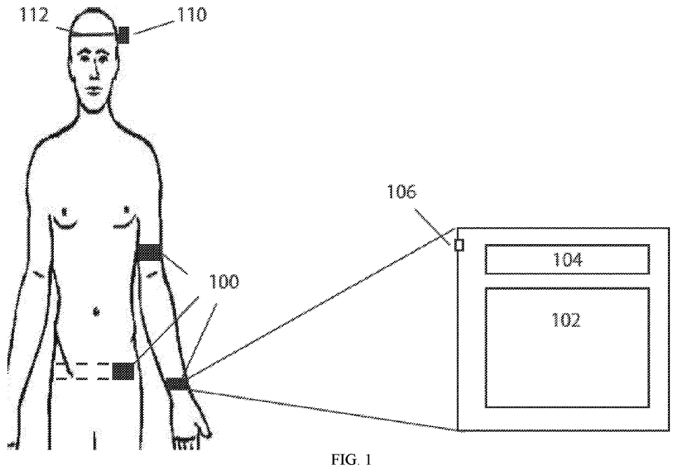

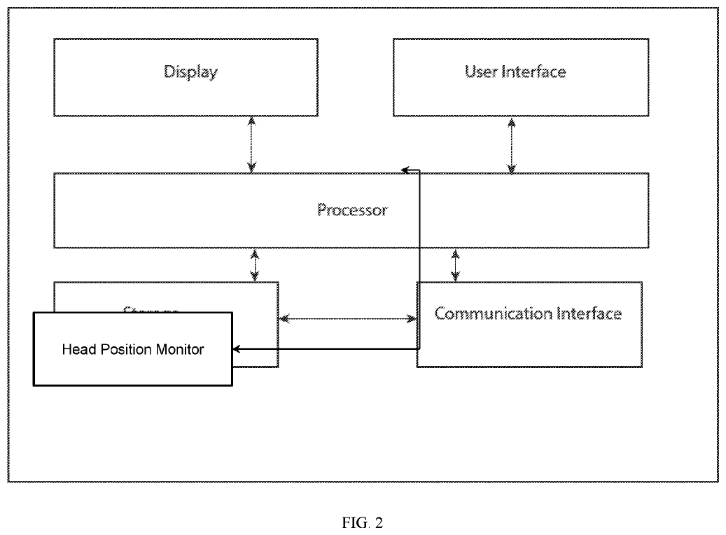

In certain aspects, referring to FIG. 1 and FIG. 2 as non-limiting examples, a head position monitoring device 112 having sensor 110 comprise a device or mechanism to removably attach to a subject's head one or more sensors 110 capable of determining the location, position, tilt, or orientation of the head. The device or mechanism to removably attach the one or more sensor 110 to a subject's head can be, but is not limited to, a headband, a helmet, a clip, an adhesive, a clamp, eye wear (e.g., goggles or glasses) etc. In some instances, the sensor 110 is capable of determining a subject's head or the sensor's position in three dimensions. In some instances, the sensor is capable of determining the subject's head or the sensor's position in two dimensions. In some instances, sensor 110 can be any device known in the art to be used to determine spatial orientation. In some instances, sensor 110 is or comprises an accelerometer. In some instances, the sensor is a 2 or 3 axis accelerometer. Accelerometers can include, but are not limited to a piezoelectric accelerometer, a capacitive accelerometer, and a multi-axis accelerometer. Non-limiting examples of piezoelectric accelerometers include accelerometers that contain microscopic piezoelectric crystals. In some instances, piezoelectric elements can induce voltage when a pressure is applied; thus, if acceleration forces causes any stress or pressure on the microscopic piezoelectric crystals the acceleration can be measured from the voltage generated. Non-limiting examples of capacitive accelerometers include accelerometers that sense a change in electrical capacitance with respect to acceleration. In some aspects, capacitive accelerometers may have structures with certain capacitance between them—if an accelerative force moves one of the structures, then the capacitance will change. Non-limiting examples of multi-axis accelerometer include 2 axis accelerometers that can measure two dimensional positioning or movement of an object and 3 axis accelerometers that can measure three dimensional positioning or movement of an object.

In some aspects, a user interface 100 can be electronically or wirelessly coupled to the head position monitor. In some instances, the display or user interface can be worn or attached to a subject. In some instances, the display or user interface is not located on or attached to the subject, but is a stand-alone device that can be positioned in the proximity of a subject during use. In some instances, the display or user interface can have a display 102, speaker 104, or push button 106. In certain aspects, the display or user interface can include a touchscreen or scroll device. In one aspect a touch screen, after being powered, can display a Main Menu that can include an INTRODUCTION, DIAGNOSTICS, and MANEUVERS selection. When ‘INTRODUCTION’ is selected, the system will explain BPPV, using voice and images. When ‘DIAGNOSTICS’ is pressed, the touch screen can display the diagnostic options that can include: Dix-Hallpike Right Ear, Preview; Dix-Hallpike Left Ear, Preview; and the like. The ‘Preview’ option will explain the procedures using voice and images, in order to familiarize the patient with the various positions and the general flow of, for example the Dix-Hallpike diagnostics.

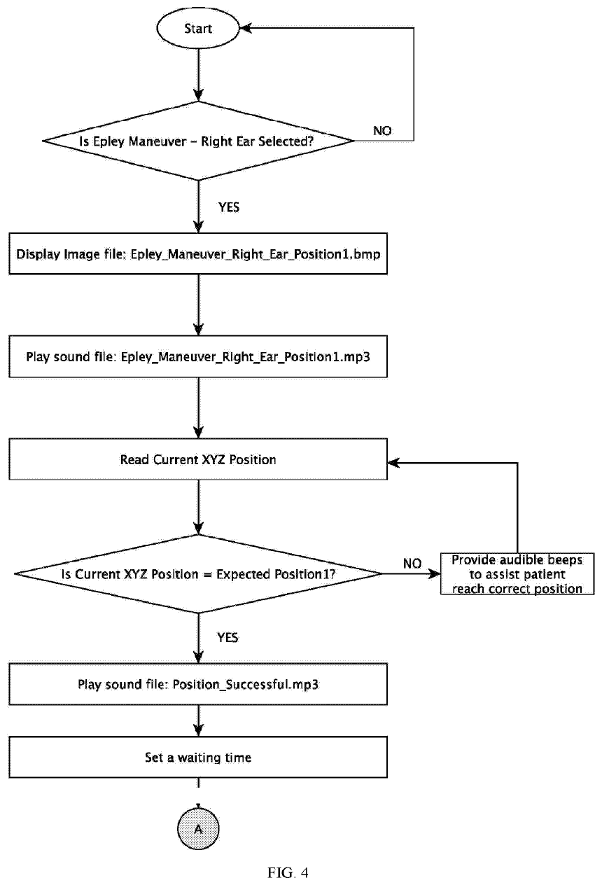

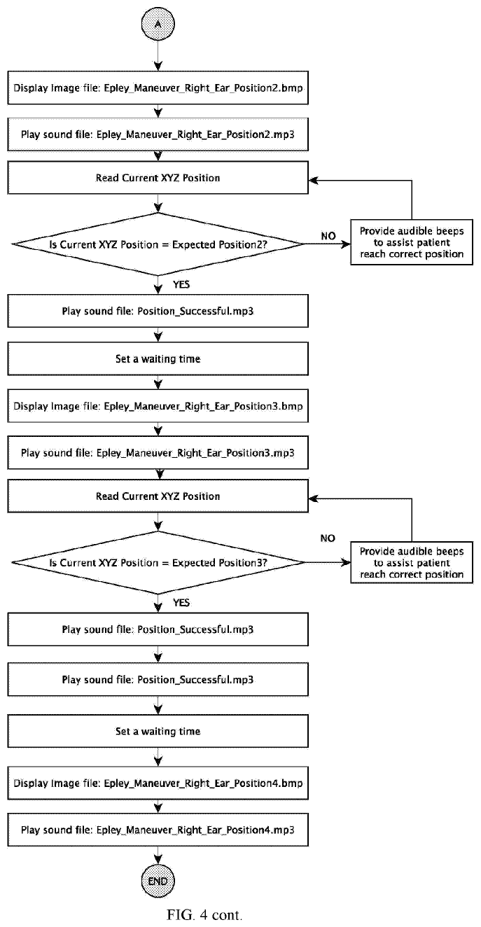

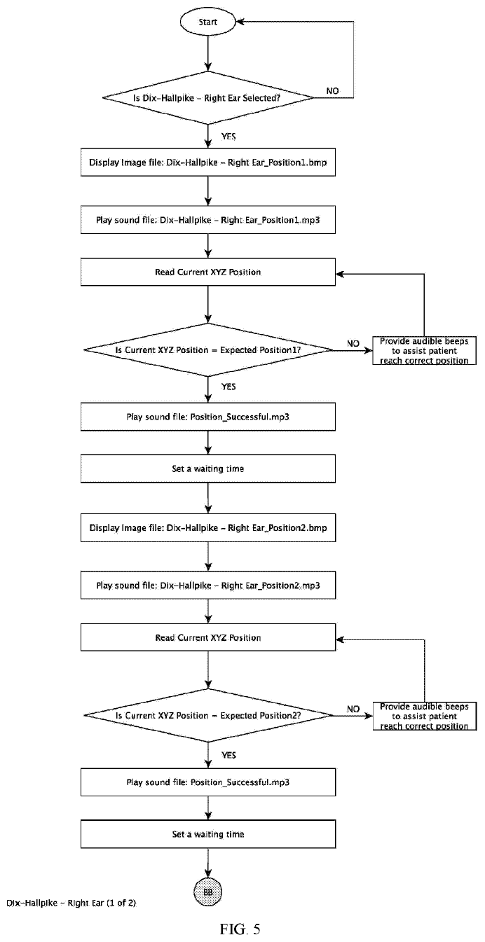

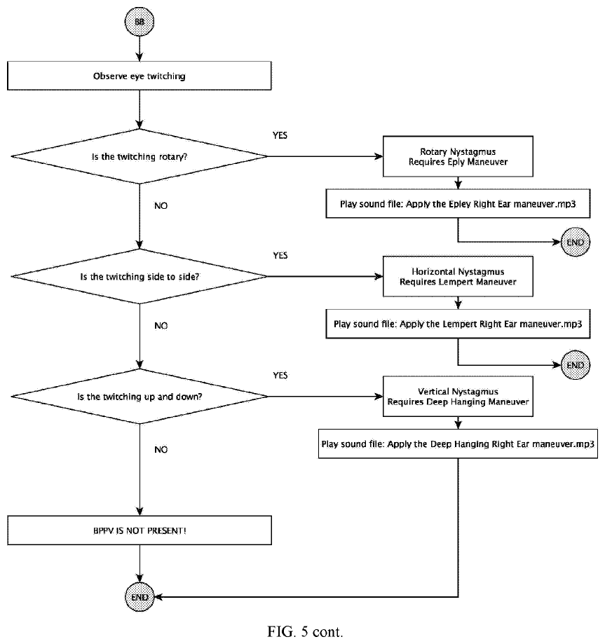

When ‘MANEUVERS’ is pressed, the touch-screen can display: “Select Right Ear” and “Select Left Ear”. When ‘Right Ear’ is pressed, the touch-screen can display a number of options, such as: EPLEY-Right, Preview; LAMPERT-Right, Preview; DEEP-HEAD-HANGING-Right, Preview, etc. The ‘Preview’ option will explain the procedures using voice and images, in order to familiarize the patient with the various positions and the general flow of the maneuvers. When ‘Left Ear’ is pressed, the touch-screen can display a number of options, such as: EPLEY-Left, Preview; LAMPERT-Left, Preview; DEEP-HEAD-HANGING-Left, Preview; etc. The ‘Preview’ option will explain the procedures using voice and images, in order to familiarize the patient with the various positions and the general flow of the maneuvers. FIG. 4 and FIG. 5 provide a scheme for programming an Epley maneuver or a Dix-Hallpike maneuver, respectively.

FIG. 4 illustrates one example of performing the Epley Maneuver. The system waits for the Epley Maneuver option to be selected. When the Epley Maneuver is selected, the system displays an image of the first position, and plays a sound file instructing the patient to move to that position as illustrated in the image displayed. The sensor that is worn by the patient transmits the current XYZ position of the patient, the microprocessor receives the data and compares it to the expected XYZ values stored in memory for a first position. If the current values for the first position do not match to the expected values (+−a certain set percentage), the system sounds audible beeps to assist the patient to achieve the right position. Once the position of the patient matches (within a set tolerance) the expected position stored in memory, the system plays a sound file announcing ‘position successful’ and instructs the patient to remain in that position until instructed again to change the position to a second position. The above process repeats until last position is reached and the maneuver is complete.

The process for subsequent positions include: the sensor transmitting the current XYZ position of the patient, the microprocessor receives the data and compares it to the expected XYZ values stored in memory for a second position. If the current values for the second position do not match to the expected values (+−a certain set percentage), the system sounds audible beeps to assist the patient to achieve the right position. Once the position of the patient matches (within a set tolerance) the expected position stored in memory, the system plays a sound file announcing ‘position successful’ and instructs the patient to remain in that position until instructed again to change the position to a third position. The sensor transmits the current XYZ position of the patient, the microprocessor receives the data and compares it to the expected XYZ values stored in memory for the third position. If the current values for the third position do not match to the expected values (+−a certain set percentage), the system sounds audible beeps to assist the patient to achieve the right position. Once the position of the patience matches (within a set tolerance) the expected position stored in memory, the system plays a sound file announcing ‘position successful’ and instructs the patient to remain in that position until instructed again to change the position to a fourth position. At the end of the fourth position, the sound file tells the patient that the maneuver is complete.

FIG. 5 illustrates a scheme for performance of the Dix-Hallpike maneuver. The system can provide a preview of the maneuver. The system waits for the Dix-Hallpike Maneuver Preview option to be selected. When the Dix-Hallpike Maneuver-Right Ear preview is selected, the system displays an image of a first position, and plays a sound file instructing the patient to move to that position as illustrated in the image displayed. After a short delay, the system displays an image of a second position, and plays a sound file instructing the patient to move to that position as illustrated in the image displayed. After a short delay, the system displays an image of third position, and plays a sound file instructing the patient to move to that position as illustrated in the image displayed. After a short delay, the system displays an image of a fourth position, and plays a sound file instructing the patient to move to that position as illustrated in the image displayed. The sound file also tells the patient that the review of the maneuver is complete.

After the review is complete the system can initiate the scheme illustrated in FIG. 5. The system waits for the Dix-Hallpike Maneuver option to be selected. When the Dix-Hallpike Maneuver is selected, the system displays an image of the first position, and plays a sound file instructing the patient to move to that position as illustrated in the image displayed. The sensor transmits the current XYZ position of the patient, the microprocessor that receives the data and compares it to the expected XYZ values stored in memory for the first position. If the current values for the first position do not match to the expected values (within a certain set percentage), the system sounds audible beeps to assist the patient to achieve the right position. Once the position of the patient matches (within a set tolerance) the expected position stored in memory, the system plays a sound file announcing ‘position successful’ and instructs the patient to remain in that position until instructed again to change the position to a second position. The sensor transmits the current XYZ position of the patient, the microprocessor receives the data and compares it to the expected XYZ values stored in memory for the second position. If the current values for the second position do not match to the expected values (within a certain set percentage), the system sounds audible beeps to assist the patient to achieve the right position. Once the position of the patience matches (within a set tolerance) the expected position stored in memory, the system plays a sound file announcing ‘position successful’. At this point the eyes are observed for fast movement-eye twitching. If a rotary twitching is observed, by a person or by a camera—the system announces that the Epley right ear maneuver should be performed. If a horizontal twitching is observed, by a person or by a camera—the system announces that the Lempert right ear maneuver should be performed. If a vertical twitching is observed, by a person or by a camera—the system announces that an the Deep Hanging right ear maneuver should be performed.

In some instances, the electronic or wireless coupling can couple the display or user interface to a processor. In some instances, the electronic or wireless coupling can couple the head position monitor to a processor. In some instances, the electronic or wireless coupling can couple the head position monitor to a data storage. In some instances, the electronic or wireless coupling can provide a communication interface that can accommodate one or more of wired communication interfaces (USB, microUSB, etc.) or wireless communication interfaces (WiFi, Bluetooth, etc.). In certain aspects, a smartphone, smartwatch, tablet, or other mobile device can be configured or programmed to integrate various aspects of the methods and process described herein. In certain aspects, a mobile application or software can be used to implement or control the methods and devices described herein.

In certain aspects at least one display is configured to indicate the head position in a head position or movement regimen that the subject is performing or attempting to perform. In certain aspects, compliance with the desired head position is displayed, such as, but not limited to by an audible indication of position relative to a target positions, proximity to compliance, and/or direction the subject's head should be moved to become compliant. In some aspects, an indicator of x and y axis, or the x, y and z axis, which will assist the patient in orienting head position.

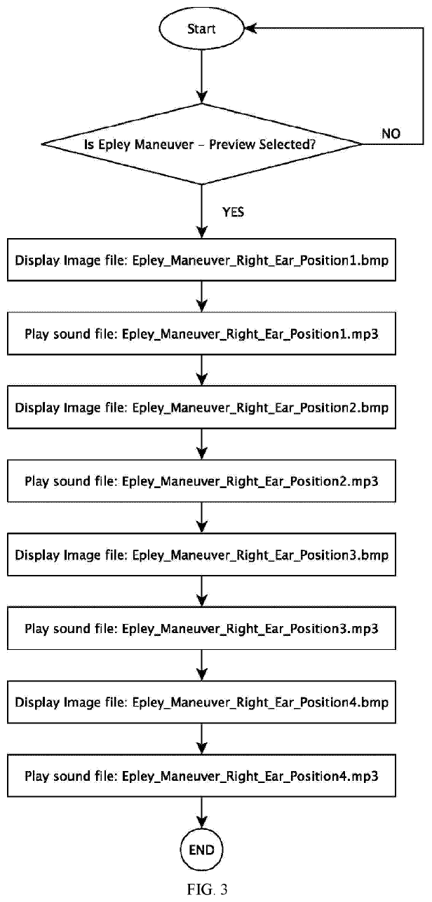

In certain aspects the system provides a preview, review, or summary of the regimen and one or more steps needed to complete the regime. FIG. 3 provides one example of a preview scheme. In one aspect, the preview scheme the system is waiting for the Epley Maneuver Preview option to be selected. When the Epley Maneuver-Right Ear preview is selected, the system displays an image of a first position, and plays a sound file instructing the patient to move to that position as illustrated in the image displayed. After a short delay, the system displays an image of a second position, and plays a sound file instructing the patient to move to that position as illustrated in the image displayed. After a short delay, the system displays an image of a third position, and plays a sound file instructing the patient to move to that position as illustrated in the image displayed. After a short delay, the system displays an image of a fourth position, and plays a sound file instructing the patient to move to that position as illustrated in the image displayed. The sound file also tells the patient when the maneuver is complete. The purpose of the Preview option is to get the patient familiar with the process, so when he goes through the actual procedure he knows what is expected of him to do.

The user display or interface can display a video or slide show of the treatment regime or desired position(s). In some aspects, the display displays a simulation of the location of the subject's head or a simulation of the direction that a subject's head should be moved to become compliant. In some instances, the display displays a simulation of what is happening in a subjects ear canal as the subject's head is moved or positioned. In certain aspects, information is provided in alphanumeric form across or in a text window of the device. In a further aspect, information is provided in a display window. The display can be configured to provide graphics. In certain embodiments the display can provide visual or audio signals related to an upcoming task (e.g., reminders), completion of a task (e.g., providing positive reinforcement), failure to complete tasks (e.g., warnings or alerts), etc. In certain aspects, a signal can be communicated to a server that is monitored by a third party, e.g., nurse or staff at a physician's office or hospital.

In certain aspects, the user interface is a touch screen display. In certain aspects, the user interface contains one or more actual or virtual button(s), switch(es), key(s), dial(s), lever(s), slide(s), etc. In certain aspects, the user interface is a touchscreen/display. In certain aspects, the user interface enables a user to program the head monitoring device or system to recognize specific head positions or movements. In certain aspects, the user interface enables a user to select from more than one treatment regimen or head position. In certain aspects, the user interface enables a user to record data, instructions, or sounds into the device, system, or a memory electronically or wirelessly coupled thereunto directly or indirectly. In some aspects, the user interface enables a user to modify a treatment regimen or head position stored in the device or system.

In certain aspects, the device, method, and/or system includes a speaker or sound system. In some aspects, the speaker or sound system provides instructions to the subject or a care provider. The instruction can be provided by any means of sound known in the art to provide instructions, such as voice commands, beeps, ascending or descending note(s), clicks, alarms, etc. In some aspects, the instructions provide directions for a head position or treatment regimen that is desired to be performed. In some aspects, the instructions provide real-time guidance to subject or a medical care provider to place the subject's head or move the subject's head in compliance with the treatment regimen or head position desired. In some aspects, the real-time guidance is provided as voice commands.

In some instances, the device can be programmed with one or a multiplicity of treatment positions or regimens. In some aspects, the device can be provided to a subject along with a multiplicity of unique identifiers that can be associated with treatments or head positions. In some instances, a database having entries representing different treatments and schedules can be queried and the appropriate records transmitted and stored on the device. Once the appropriate data is installed on the device the medical care professional can modify parameters as needed for a subject—for example a health care provider programs a particular treatment or head position for a particular period of time, or a particular treatment to be applied on a particular schedule.

One non-limiting example of a control process for a programming and using a monitoring device or system includes, initiating programming for a treatment regimen or head position into the device and/or system, the user places the sensor on the subject's head and positions the subject's head in the first desired position. In some instances, the user then provides an input to the user interface (activates the user interface) and in response the device or system displays or indicates that a first head position is to be measured, the device or system reads the sensor output and then saves the sensor outputs as position 1 in the memory. In some aspects, a second position can then be programmed into the device or system by activating the user interface, and in response the device displays an indication that a second head position is to be measured, the device or system reads the sensor, and then saves the sensor outputs as position 2 in the memory. This process can be repeated for programming additional head positions into the device and/or system. In some instances, 1, 2, 3, 4, 5, 6, 7, 8, 9, 10 or more positions can be saved individually or as part of a regimen. In some instances, 1, 2, 3, 4, 5, 6, 7, 8, 9, 10 or more regimens of one or more positions can be saved.

In some aspects, to initiate monitoring of compliance with a treatment regime involving positioning or movement of a subject's head or initiating providing instruction to a subject or a medical care provider regarding positioning of a subjects head to be compliant with a desired head position, the user activates the user interface and in response the device displays or provides an indication that a first head position is desired, the device and/or system reads from the memory the sensor output saved as position 1, and activates the display or sound system to indicate any one, combination, or all of the following: the real-time position of the subjects head or head monitoring device; if the subjects head or the head monitoring device is in the same or a similar position to the saved position 1; or the direction the subject's head and/or the head monitoring device needs to move to be in the same or similar position to the saved position 1. In some aspects, once the head and/or head monitoring device is positioned in the same and or a similar position to saved position 1, the device or system then sets a timer period wherein if the head position is retained in the same or a similar position for the extent of the timer period, the display or sound system indicates that the subject has completed the task or should move their head to a second position. In other aspects, once the head or head monitoring device is positioned in the same or a similar position to saved position 1, the display or sound system indicates that the subject has completed the task or should move their head to a second position. In some aspects, the device or system then reads from the memory the sensor output saved as position 2, and activates the display or sound system to indicate any one, combination, or all of the following: the real-time position of the subject's head or head monitoring device; if the subject's head or head monitoring device is in the same or a similar position to the saved position 2; or the direction the subject's head and/or the head monitoring device needs to move to be in the same or similar position to the saved position 2. In some aspects, once the head or head monitoring device is positioned in the same and or a similar position to saved position 2, the device or system then sets a timer period wherein if the head position is retained in the same or a similar position for the extent of the timer period, the display or sound system indicates that the subject has completed the task or should move their head to a third position. In other aspects, once the head or head monitoring device is positioned in the same or a similar position to saved position 2, the display or sound system indicates that the subject has completed the task or should move their head to a third position. This process can be repeated for any additional head positions saved in the device or system. In some instances, 1, 2, 3, 4, 5, 6, 7, 8, 9, 10 or more positions can be used or selected individually or as part of a regimen. In some instances, 1, 2, 3, 4, 5, 6, 7, 8, 9, 10 or more regimens of one or more positions can be used or selected.

In some instances, at the time a task is scheduled the subject can receive a visual or audio indicator that a treatment or a specific head position needs to be performed. In certain aspects, the visual or audio indicator correlates with a unique identifier for the treatment. For a non-limiting example, if a particular treatment is due and the treatment has been given a unique identifier, such as the word “blue” the device will display a blue color, the word “blue”, or will say “blue”. Thus, informing the subject that it is time to perform the treatment associated with the blue unique identifier.

In certain aspects, the device or system is capable of displaying the type of treatment, and the time of the day a subject needs to perform a certain treatment. In some aspects, the treatment will be determined and prescribed by a physician or other qualified medical personnel. In certain aspects, the qualified medical personnel can program or have the device programmed for 1, 2, 3, 4, 5, or more schedules. In some instances, every time the patient completes a task, e.g., a displayed treatment, he or she confirms completion of the task by activating a user interface of the device. In some instances, activation of the user interface records the event along with the date, the time, and the specific type treatment via the processor into data storage. In some aspects, the completion or lack of completion of a task will be used to update the status of the device. In certain aspects, the status of the device will be displayed periodically. For a non-limiting example, after completion of a task the display can shut off or go dark until time to display the next task. In some instances, this process repeats itself throughout the day based on the schedule programmed. In further aspects, the device cycles on a 24-hour clock and starts a new cycle every 24 hours.

In some aspects, when the subject visits the physician, the data in storage in the device or system can be retrieved, e.g., the device may be plugged into a computer and data transferred. The physician then can see all the recorded data and may save the data to the patient's medical file. In some instances, the data shows the physician if the patient followed the directions and helps the physician to evaluate the patient's condition.

In certain embodiments, the display can be a LCD screen or external display such as a smart phone or tablet communicating with the device using technologies such as BLUETOOTH™.

In certain embodiments the devices and methods disclosed herein can be coupled to other sensors or monitors.

Another non-limiting example is of a routine for displaying a head position in the x or y axis, respectively. In certain aspects, the device or system will monitor head position and provide an audio indicator (e.g. beep or vocal command), visual indicator (e.g. lights, figure, or diagram), tactile indicator (e.g. vibration or pulses), or a combination thereof, the indicator relating to the relationship between the current head position and the desired saved head position setting. In other aspects, the indicators can indicate an acceptable head position or one that is within a range of acceptable positions relative to the desired saved head position, an unacceptable head position, or one that may be detrimental to the treatment outcome, or an intermediate head position in that it is a position that is acceptable but not ideal in regard to fostering a positive treatment outcome. The routines can query the head position and respond with an appropriate indicator, which is provided as a visual indicator in the examples provided. In the non-limiting examples provided, a head position within 5 degrees of the desired saved head position setting is an acceptable head position, a head position within 10 degrees and a greater than 5 degrees of the desired saved head position is an intermediate position, and a head position within 15 degrees and greater than 10 degrees is an unacceptable position. In these non-limiting examples, any position over 15 degrees will trigger an elevated response to bring the head position to the patient's or medical personnel's attention. In these non-limiting examples, the device will log various parameters associated with each head position query to a memory device or transmit it to a server. The parameters can include, but are not limited to head position, time, duration, etc.

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Using the Device and/or System in Diagnosis of BPPV

As a non-limiting example, the device or systems disclosed herein can be used to assist in the diagnosis of Benign Paroxysmal Positional Vertigo (BPPV).

BPPV is diagnosed using the Dix-Hallpike maneuver on the patient. The diagnosis of the type of BPPV and the type of maneuver that needs to be applied is determined by observing eye twitches (nystagmus) of the patient during the Dix-Hallpike maneuver. Eye twitching must be present for BPPV to be diagnosed. The Dix-Hallpike test is performed with the patient sitting upright on the examination table with the legs extended. The patient's head is then rotated to one side by approximately 45 degrees. The clinician helps the patient to lie down backwards quickly with the head held in approximately 20 degrees of extension. This extension may either be achieved by having the clinician supporting the head as it hangs off the table or by placing a pillow under their upper back. The patient's eyes are then observed for about 45 seconds as there is a characteristic 5-10 second period of latency prior to the onset of nystagmus. If rotational nystagmus occurs then the test is considered positive for benign positional vertigo. During a positive test, the fast phase of the rotatory nystagmus is toward the affected ear, which is the ear closer to the ground. The direction of the fast phase is defined by the rotation of the top of the eye, either clockwise or counter-clockwise.

The subject wears the device or system disclosed herein that is programmed or has been programmed for the positions required for the Dix-Hallpike maneuver. In this way, the patient or the medical care provider may be informed if the head position of the patient is correct, and assistance by the medical care provider to keep the patients head in the correct position can be minimized or eliminated because of the devices or system's informing the patient how to keep the head in the correct position. In this way, the medical care provider is able to devote more attention to carefully observe and characterize any nystagmus.

The following are the types of nystagmus that may be observed using the Dix-Hallpike maneuver and the head movement treatments to be performed to correct the BPPV: rotatory nystagmus indicates Posterior canal BPPV for which the Epley maneuver is prescribed; lateral nystagmus indicates Lateral canal BPPV for which Lempert maneuver is prescribed; and vertical nystagmus indicates Superior canal BPPV for which The Deep Hanging maneuver is prescribed.

### Example 2

### Using the Device or System in Diagnosis or Treatment of BPPV

While experiencing a vertigo episode, a subject's eyes twitch and the subject is often dizzy. These conditions make it very difficult to follow visual instructions in order to figure out all the steps, positions, angles, and timing needed to complete a treatment maneuver.

In one embodiment, the device or system disclosed herein includes a voice system and screen display, such as, but not limited to Thin Film Transistor (“TFT”) color touch screen. Before any test or maneuver, the patient watches a video or a slideshow on one of the two screens to become familiar with the procedure about to be performed. Alternatively or simultaneously audio instructions can also be given to the patient.

The patient wears a head position monitor connected to the system disclosed herein. The head position monitor can be, but is not limited to, a sports headband or eyewear that includes a sensor or camera.

For diagnosis, or to evaluate the condition of the patient, Dix-Hallpike diagnostic maneuver is selected on the device or system disclosed herein. A video or slideshow is shown to the patient on at least one of the two display screens that shows a complete Dix-Hallpike diagnostic maneuver and explains the various steps, angles, and timing.

When the patient is ready to start, the voice system will prepare the patient before every step that has to take place. The patient first listens to what is going to take place and then the voice announces the instruction that needs to be executed by the patient. The voice keeps guiding the patient in real-time, based on the feedback the system disclosed herein receives from the sensors on the patient's head. The voice guidance simulates physician or therapist instruction to the patient.

As the patient goes through the steps of the maneuver a second display screen can show a simulation of what is happening in the ear canals as the patient is moving their head and body in different angles. The simulation is based on the calculated angles of the Superior, Posterior, and Horizontal canals in the patient as the head is moving. The position of the floating crystals that cause BPPV are affected by gravity; thus, when the patient's head stops moving, the position of the crystals can be predicted. In some instances, the whole process is recorded and analyzed later, comparing the head movement to the resulting simulation.

The same procedure can be performed for an appropriate treatment regimen for treatment of the patient's BPPV, such as for an Epley maneuver, a Lempert maneuver, and/or The Deep Hanging maneuver.

1. A device for monitoring the spatial orientation of a part of a subject's body comprising:
a sensor capable of determining the one, two, or three dimensional spatial orientations of the sensor and capable of being removably attached to the part of the subjects body;
data storage capable of storing more than one spatial orientation of the sensor under an unique identifier;
a processor capable of determining a difference between a real-time spatial orientation of the sensor and a stored orientation of the sensor;
a user interface capable of receiving input from a user or programmer comprising
(i) a target position that is defined when the spatial orientation of the sensor is to be stored in the storage under a unique identifier for the stored orientation, and
(ii) when the difference is to be determined by the processor between the real-time spatial orientation of the sensor and the stored orientation of the sensor;
a speaker or sound system capable of producing sounds to indicate if there is or is not a difference in the real-time spatial orientation of the sensor and the stored orientation of the sensor;
a display capable of displaying real time spatial orientation or the target position;
wherein the sensor is capable of electronically or wirelessly coupling to the storage, the processor, the user interface, the sound system, and the display directly or indirectly.

2. The device of claim 1, further comprising wherein the display is capable of displaying an indication if there is or is not a difference in the real-time spatial orientation of the sensor and the target position.

3. The device of claim 1, further comprising wherein the sound system or display is capable of indicating the direction the body part needs to move to be within a certain real-time position of the sensor relative to the target position.

4. The device of claim 1, wherein more than one spatial orientations of the sensor are stored under unique identifiers and are capable of being stored as sequential orientations to be achieved in a regimen stored in the memory.

5. The device of claim 4, wherein:
the sound system or display is capable of indicating that the sensor real-time position is within a certain range of the target position; and
the sound system or display is capable of then, or at the same time, indicating the direction the body part needs to move to be within a certain range of a second target position of the regimen.

6. The device of claim 1, further comprising a timer capable of timing the amount of time the sensor real-time position is within a certain range of the target position.

7. The device of claim 1, wherein the device is capable of storing data regarding if or when the sensor real-time position was within a range of a certain target position.

8. The device of claim 1, wherein the device is capable of transmitting data regarding if or when the sensor real-time position were within a range of certain target position.

9. The device of claim 1, wherein the device is capable of displaying a representation of the orientation of the body part needed to place the sensor within a target position.

10. The device of claim 1, wherein the sound system is capable of producing voice commands to indicate if there is or is not a difference in the real-time spatial orientation of the sensor and the stored orientation of the sensor.

11. The device of claim 1, wherein the stored orientation of the sensor under a unique identifier corresponds with a position of the body part that is to be achieved during a medical treatment or diagnostic.

12. The device of claim 1, wherein the medical treatment or diagnostic is to be performed outside of the real-time supervision of a medical care provider.

13. The device of claim 1, wherein the part of the subject's body is the subject's head.

14. The device of claim 13, wherein the medical treatment or diagnostic is to treat or diagnose vertigo in the subject.

15. A system for tracking the orientation of a subject's body part, the system comprising:
a sensor capable of determining the one, two, or three dimensional spatial orientation of the sensor and capable of being removably attached to the part of the subjects body;
a data storage capable of storing more than one spatial orientation of the sensor under unique identifier;
a processor capable of determining a difference between the real-time spatial orientation of the sensor and a stored orientation of the sensor;
a user interface capable of receiving input from a user or programmer comprising
(i) when the spatial orientation of the sensor is to be stored in the storage under a unique identifier for the stored orientation, and
(ii) when the difference is to be determined by the processor between the real-time spatial orientation of the sensor and the stored orientation of the sensor(s) stored under a unique identifier;
a speaker or sound system(s) capable of producing sound to indicate if there is or is not a difference in the real-time spatial orientation of the sensor and the stored orientation of the sensor under a unique identifier;
a display;
wherein the sensor is capable of electronically or wirelessly coupling to the data storage, the processor, the user interface, the sound system, and the display directly or indirectly.

16. The system of claim 15, wherein the display is capable of displaying an indication if there is or is not a difference in the real-time spatial orientation of the sensor and the stored orientation of the sensor under a unique identifier.

17. The system of claim 15, wherein the sound system or display is capable of indicating the direction the body part needs to move to be within a certain range of the stored orientation of the sensor.

18. The system of claim 15, wherein more than one spatial orientations of the sensor is stored under unique identifiers and are capable of being stored as sequential orientations to be achieved in a regimen stored in the memory under unique regimen identifier.

19. The system of claim 18, wherein the sound system or display is capable of indicating that the sensor real-time position is within a certain range of the stored orientation of the sensor stored under a unique identifier within a stored regimen stored under a unique regimen identifier; and wherein the sound system or display is capable of then, or at the same time, indicating the direction the body part needs to move to be within a certain range of the next sequential orientation of the sensor stored under a unique identifier in the regimen stored under a unique regimen identifier.

20.-33. (canceled)

34. A method of monitoring the position of a subject's body part relative to at least one pre-programmed desired orientation of the subject's body part by using any one of the devices claim 1, the method comprising in any order:
removably attaching a sensor on the subject's body part to be monitored;
electronically or wirelessly coupling the sensor directly or indirectly to the processor, storage, and user interface, wherein the storage have been preprogrammed to contain at least one orientation of the sensor associated with a desired orientation of the subject's body part;
optionally using the user interface to select the pre-programmed desired orientation of the sensor associated with a desired orientation of the subject's body part to be used by the processor in determining the difference between the real-time spatial orientation of the sensor and the pre-programmed desired orientation of the sensor;
electronically or wirelessly coupling the processor directly or indirectly to at least one display and/or at least one sound system so that it indicates if there is or is not a difference in the real-time spatial orientation of the sensor and the pre-programmed desired orientation of the sensor;
wherein monitoring indication from the display or sound system provides information regarding the position of a subject's body part relative to the at least one pre-programmed desired orientation of the subject's body part.

35.-40. (canceled)
